(19) United States
(12) Patent Application Publication
(10) Pub. No.: US 2020/0315655 A1
(43) Pub. Date: Oct. 8, 2020

(54) METHOD AND APPARATUS FOR A MEDICAL GUIDANCE DEVICE

(71) Applicant: Canon U.S.A., Inc., Melville, NY (US)

(72) Inventors: Erin McLean Bitner, Waltham, MA (US); Morgan Carlson Rudolph, Nashua, NH (US); Katherine F. MacNamee, Litchfield, NH (US); Ronald C. Kurz, JR., Yorktown, VA (US); Lydia G. Olson, Swampscott, MA (US); Charles George Hwang, Wellesley, MA (US)

(21) Appl. No.: 16/838,851

(22) Filed: Apr. 2, 2020

Related U.S. Application Data
(60) Provisional application No. 62/831,042, filed on Apr. 8, 2019.

(51) Int. Cl. A61B 17/34

(52) U.S. Cl. CPC A61B 17/3403; A61B 2017/00902

(57) ABSTRACT

Medical guidance device capable of improving precision and accuracy of medical surgery by ensuring accurate and consistent placement of the medical guidance device, while further allowing for repeated use of the medical guidance device in a single setting.

### CROSS REFERENCE TO RELATED PATENT APPLICATIONS

This application claims priority from U.S. Provisional Patent Application No. 62/831,042 filed on Apr. 8, 2019, in the United States Patent and Trademark Office, the disclosure of which is incorporated herein in its entirety by reference.

### FIELD OF THE DISCLOSURE

The present disclosure relates generally to apparatus and methods for a medical guidance device and, more particularly, to a medical guidance device for a needle, capable of adaptation and repeated secure adhesion to various surface shapes.

### BACKGROUND OF THE DISCLOSURE

Accurate and precise manipulation and orientation of medical tools is a critical factor in the successful use of these tools in surgical procedures. In particular, the accurate and precise orientation of a medical tool is especially critical in the insertion of a needle-like tool according to plan based on medical images, such as computed tomography (CT) and Magnetic Resonance Imaging (MRI) in percutaneous interventions.

The general purpose of any needle guidance device is to direct the needle onto a path passing precisely through a selected insertion point to the target location within the patient's anatomy. This objective involves limiting the position and orientation to one azimuthal angle and one angle of elevation with respect to a selected insertion point upon the patient's skin surface. Selection of an insertion point, and both azimuthal and elevation angles, defines the needle trajectory into the patient's anatomy. The needle is able to slide axially along the chosen trajectory, to the desired depth.

There are examples of needle guidance devices currently available on the market that incorporate adhesive mounts to attach to the patient's skin, and are adjustable so that the physician can choose a needle trajectory within some range. By way of example, United States Patent Publication No. 2014/0336670, and United States Patent Publication No. 2008/0200798, both disclose needle guidance devices that claim to achieve these objectives. Both devices provide a means to set a needle trajectory and claim to support the needle during partial insertions, and both devices have an adhesive layer under the base of the device to adhere the device to the patient skin surface.

Devices with adhesive pads generally have easy pull tabs that require the adhesive to face the user when the liner is being removed. The device will then be turned away from the user for placement. Easy pull tabs may be liners that are folded back on itself, segmented liners that overlap, or liners that extend over the edge of the adhesive. These variations all achieve a section or sections of liner that do not have adhesive underneath so the user may easily locate and remove the liner.

Examples of existing disclosure in the art include U.S. Pat. No. 8,790,311, which describes an adhesive pad intended to mount a medical guidance device onto a patient. The mounting pad has an opening in the center for viewing the target location. The release liner is divided into two or more sections which are all connected to the device's packaging. With a single upward motion, the device can be lifted out of it's packaging. This motion will remove the liner form the adhesive.

Another exemplary disclosure is U.S. patent publication no. 2016/0367789. This publication describes an adhesive pad intended to secure a catheter that has been placed into a patient. The adhesive creates a seal around a transparent window for viewing the catheter insertion point and containing any blood. The pad also has a securement member for holding the distal end of the catheter steady.

However, existing mounted medical devices require precision placement to a target location, and the associated adhesive pad designs require the user to remove the lining prior to aligning and affixing the device. This allows the end user one opportunity to place the device in the correct location, all the while, ensuring the adhesive remains unsoiled, to ensure proper adhesion. In addition, such devices tend to block the user's view when being aligned and affixed, which further exacerbates the chances of user error in placement.

Furthermore, alternative existing mounted devices having reusable adhesives are limited in the number of times they may be used on a patient. The majority of adhesives see a significant reduction in effectivity (hold strength) with each use. Additionally, the adhesive has the potential to pick up dirt, debris, or contaminants with each placement. If the device must be removed and re-positioned, the risk of contamination increases and the quality of the adhesion is further degraded.

Accordingly, it is particularly beneficial to disclose a medical guidance device and method addressing these shortcomings, and advancing the technology to yield a stable and secure method and device, allowing for more accurate and precise mounting of the device with respect to the patient.

### SUMMARY

The present disclosure teaches a medical guiding device and methods for utilizing a medical guidance device. The medical guidance device incorporates a mountable medical guiding device comprising: an adhesive pad having at least two sections of adhesive; and at least two tabs each releasable attached to the adhesive pad with each tab extending distally from the base, wherein each of the at least two tabs may be removed for exposing each of the at least two sections of adhesive for attaching the medical guiding device, and wherein each of the at least two tabs are removable while the medical guiding device is being held in a mounting position on a subject.

In other embodiments, the medical guidance device further comprises a transparent target for aligning the device with a target on the subject.

In other embodiments, the adhesive for each of at least two tabs is removably attachable to the adhesive pad.

Furthermore, the at least two tabs may be divided into at least two segments, and each of the two tabs may be removed and/or replaced from the medical guiding device. In addition, the two tabs may have adhesive on both surfaces of the tab.

In other embodiments, the medical guidance device may further comprise a guide removably attached to the medical guiding device, or a guide permanently.

In yet additional embodiments, the base of the medical guidance device comprises a rigid ring that provides structural support to the medical guiding device.

In other embodiments, the transparent target includes a visual or mechanical element. Furthermore, the transparent target may be removable and/or pierceable.

In addition, the subject innovation also discloses a medical guiding device comprising: an adhesive pad; a guidance portion configured to guide a medical instrument; and at least two tabs each releasable attached to the adhesive pad and each extending distally from the adhesive pad, wherein each of the at least two tabs may be removed for exposing an adhesive for attaching the device, wherein each of the at least two tabs is segmented, allowing for partial or complete exposure of the adhesive while the medical guiding device is held in a mounting position on the patient.

In further disclosures, the subject innovation teaches a method for guiding a medical instrument, the method comprising: providing a medical guiding device comprising: an adhesive pad; a guidance portion; and at least two tabs each releasable attached to the adhesive pad and each extending distally from the adhesive pad, wherein each of the at least two tabs may be removed for exposing an adhesive for attaching the device, and wherein at least one of the at least two tabs is segmented, allowing for partial or complete exposure of the adhesive, holding the medical guidance device at a target location on a patient; mounting the medical guidance device at the target location by removing at least one segment of the at least two tabs without substantially moving the medical guiding device; and guiding a medical instrument to a position from the target location.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIGS. 1a and 1b provide perspective views of a medical guidance device, with FIG. 1b being an exploded view of FIG. 1a, both according to one or more embodiments of the present subject matter.

FIG. 2 is a top perspective of a medical guidance device, according to one or more embodiments of the present subject matter.

FIGS. 3a and 3b provide perspective views of a medical guidance device, namely the lobed adhesion segments, according to one or more embodiments of the present subject matter.

FIG. 4 illustrates a top perspective view of a medical guidance device, according to one or more embodiments of the present subject matter.

FIG. 5 provides a top perspective view of a medical guidance device, according to one or more embodiments of the present subject matter.

FIG. 6 illustrates a top perspective view of a medical guidance device, according to one or more embodiments of the present subject matter.

FIGS. 7a and 7b display a bottom and top perspective views of a medical guidance device, respectively, according to one or more embodiments of the present subject matter.

FIGS. 8a-8c are planar views of an element of the subject medical guidance device, namely the adhesive lobes, according to one or more embodiments of the present subject matter.

FIG. 9 illustrates a top perspective view of a medical guidance device, according to one or more embodiments of the present subject matter.

FIGS. 10a and 10b provides top perspective views of a medical guidance device, according to one or more embodiments of the present subject matter.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description and/or illustration to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated. The term “position” or “positioning” should be understood as including both spatial position and angular orientation.

In the following description, reference is made to the accompanying drawings which are illustrations of embodiments in which the disclosed invention may be practiced. It is to be understood, however, that those skilled in the art may develop other structural and functional modifications without departing from the novelty and scope of the instant disclosure.

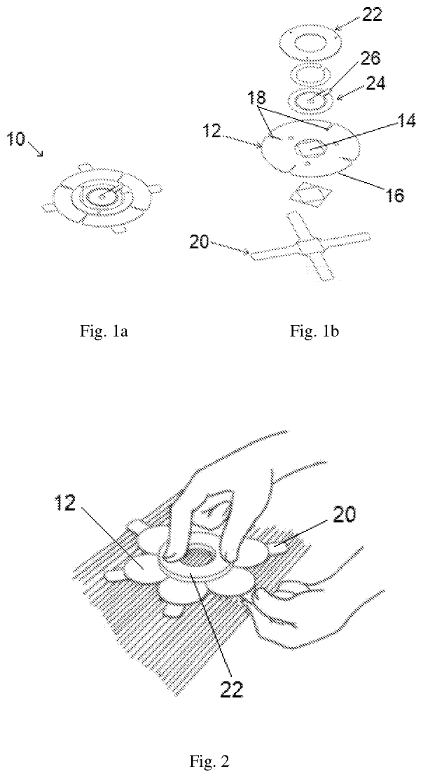

The present disclosure details a medical guidance device 10 capable of attachment to an end user, and designed to facilitate the precise placement of a medical guidance device upon and/or into the end user for inserting or otherwise attaching a medical tool upon an end user or patient. FIG. 1a provides a perspective view of the medical guidance device 10, with FIG. 1b illustrating an exploded view of the same guidance device 10 in FIG. 1a. The medical guidance device 10 incorporates an adhesive pad 12 with an opening 14. The opening 14 may be in the center of the adhesive pad 12 or in any known location required by the guidance device 10 being placed. In one embodiment, the opening 14 is an oval or circular opening substantially in the center of the medical guidance device 10 and of a size sufficient for a user to access a target position on a patient for, for example, a biopsy or ablation procedure.

The adhesive pad 12 comprises of adhesive 16 on the surface of the adhesive pad 12 coming in contact with the patent. The adhesive 16 may be found on a portion of the adhesive pad 12, or encompass the entire surface of the adhesive pad 12. In the subject embodiment illustrated in FIG. 1b, the adhesive 16 is located on a portion of the adhesive pad 12, in the shape of a cross. The adhesive pad 12 may be perforated and/or slotted 18 to create two or more sections. FIG. 1b shows an embodiment with four sections for reference. A number of tabs 20 equal the number of adhesive 16 and extend releasably past the edge of the adhesive 16 so they are accessible by the user when face down, as can been seen FIG. 2. The tabs 20 also fold back upon themselves. The tabs 20 are formed of a material that is able to bend backwards such that it can be pulled out between a patient's skin and the base of the guidance device 10, while the guidance device 10 is positioned upon the patient.

The adhesive pad 12 is oriented such that the tab 20 and adhesive 16 are facing downward with respect to the guide 22 being in contact with the adhesive pad 12, located on the opposing side. Between the guide 22 and the adhesive pad 12 is a transparent target 24 with an opening in the location of alignment onto the patient. The transparent target 24 is perforated along the edge of the adhesive pad 12 opening 14 with a pull tab 26. Pulling the pull tab 26 of the transparent target 24, will result in removal of the transparent target 24, allowing greater access to the patient.

In operating the subject medical guidance device 10, an end user mounts the guidance device 10 on a patient having a target position marked on the patient's body. This is done by first aligning the transparent target 24 with the target position on the patient. The guidance device 10 is then placed on the patient with the tabs 20 facing the patient's skin. Applying light pressure on the medical guidance device 20, and consequently pressing the adhesive pad 12 upon the patient to maintain the location, the end user pulls the tabs 20 to reveal the adhesive 16, and adhere the medical device 10 to the patient. As shown in FIG. 2, the end user is able to pull the tabs 20 after placement without having to move or tilt the medical guidance device 10 to access the adhesive 16. Thus, the positioning of the medical guidance device 10 (or base portion thereof) can be accomplished after placement. As the adhesive 16 is revealed, it will adhere to the patient's skin without substantially moving the guidance device 10. The transparent target 24 may be removed at this time if the end user requires greater access to the target area.

Thus, the guidance device 10 can be removed after targeting, and further can be repositioning at a second target site. This is particularly important for uses such as ablation or biopsy when the tumor or other target is such a size or shape that more than one skin entry point is required.

In addition, the end user may utilize a segmented tab 20 to achieve two or more placements of the guidance device 10. The end user may choose to only remove a subset of the tab 20, exposing a portion of the adhesive 16. The subset must be chosen such that the exposed adhesive 16 can adequately hold the guidance device 10 in place. Once the guidance device 10 has been placed and used, the end user may remove the guidance device 10 from its location on the patient. The used portion of the adhesive 16 may be folded back upon itself or left static. The guidance device 10 may then be utilized a second time, at a different time and/or patient, upon a secondary location, wherein the guidance device 10 is held in place with light pressure as the remaining tab 20 is removed and exposes the remaining adhesive 16, adhering the guidance device 10 to the secondary location. If two locations are planned, the end user may choose to keep the transparent target 24 intact to maintain optimal accuracy in both placements.

The subject embodiment may be manufactured by die cutting the adhesive pad 12 and it's adhesive 16 and tabs 20, or equivalent fabrication process. The adhesive pad 12 can then be perforated along the segment lines. Tabs 20 will be adhered to the adhesive pad 12 through adhesive. The adhesive pad 12 will connect to the transparent target 24 by adhesive or heat bonding. The full assembly may be adhered to the device through adhesive, heat bonding, or mechanical connectors.

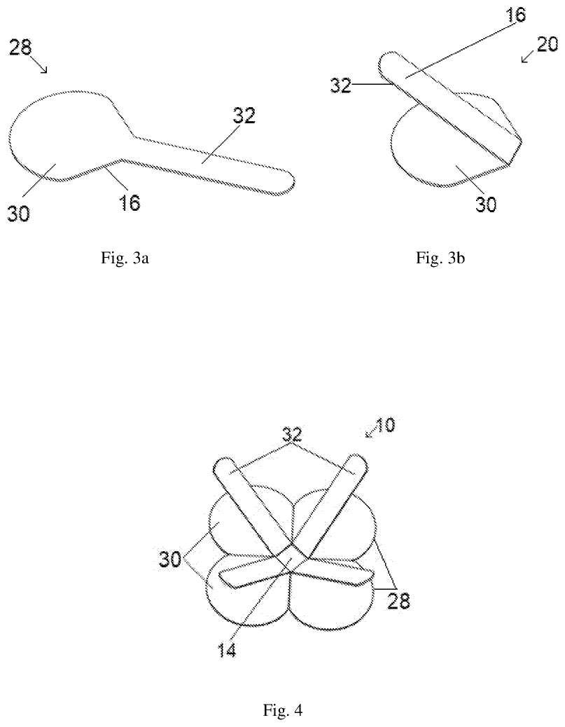

In one embodiment of the subject guidance device 10, shown in FIGS. 3a and 3b, the adhesive pad 12 is split into fully separate lobes 28. The adhesive 16 may be present in the fanned area 30 and not extend into the arm 32. The lobes 28 may vary in size and shape to accommodate the desired mounting location upon the patient's skin. Two or more lobes 28 may be aligned around the opening 14. An example utilizing 4 lobes is shown in FIG. 4, with the opening 14.

In another embodiment, only a subset of the lobes 28 may be revealed for the initial placement. This subset will be sufficient to adhere the guidance device 10 to the patient. The tabs 20 on the remaining lobes 28 may remain intact. The device may be used and removed from the patient's skin, multiple times using the other lobes 28. The tabs 20 on the remaining lobes 28 may be removed, exposing fresh adhesive 16. Accordingly, a second or multiple placement(s) of the device 10 can be achieved.

In yet another embodiment, a base including a rigid ring 34 is adhered to the upper portion of the adhesive pad 12 opposite the patient contacting face. The rigid ring 34 will allow the user to apply light pressure to the adhesive pad 12, holding it in place during removal of the tabs 20, without deforming or displacing the guidance device 10. The rigid ring 34, may be adhered to the adhesive pad 12 through adhesive, hardware such as screws, or a process such as heat sealing. The rigid ring 34 may act as a base of the medical guidance device 10 and may be of any shape but preferably does not block the opening 14. The rigid ring 34 may consist of the rigid component where the guidance portion of the medical guidance device 10 is attached directly to, and, in some cases, is permanently bonded to the rigid ring 34. Alternatively, the rigid ring 34 is configured to connect the guidance portion of the medical guidance device 10 after the adhesive pad 12 is in place on a patient. In some examples, there are mating features that are used to connect the rigid ring 34 to the guidance device 10, including, for examples, snaps, adhesives, threads, keyways, etc.

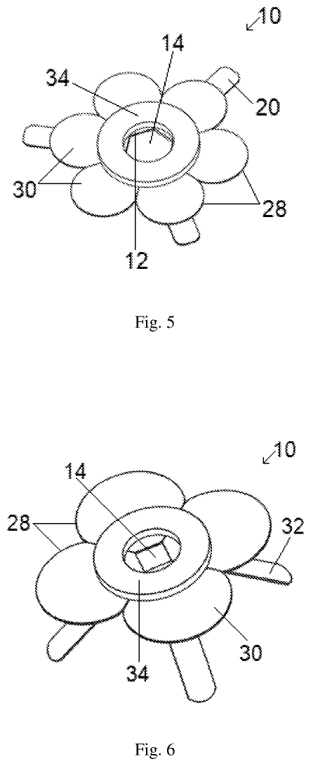

FIG. 5 is an illustration of this embodiment with the rigid ring 34 component. In embodiments where the rigid ring 34 is detachable from the guiding portion 10, a particular advantage is that the user can use the rigid ring 34 for ease of placement, alignment, and adhesion to the patient at the target area.

In some embodiments, the guidance device 10 may comprise of multiple adhesion pads 12, such that one adhesion pad 12 may be attached, used, and then removed and a second adhesion pad 12 (e.g., for guiding a different type of tool) is attached to the rigid ring 34 ready for attachment to the patient.

In another embodiment, the rigid ring 34 is adhered to the adhesive pad 12 with a temporary adhesive. After initial placement, the used adhesive lobe 28 may be fully removed from the rigid ring 34 or guidance device 10, leaving only the fresh adhesive lobes 28 for the second placement upon the patient.

In another embodiment, the adhesive 16 in the single piece adhesive pad 12 or the individual lobes 28, are double sided with the patient facing side having a tabs 20 as described previously. The non-patient facing side will have a perforation in a release liner 36 whose dimensions correspond with the surface area of the guidance device 10 or rigid ring 34. The perforated release liner 36 will have a tab 38 for ease of removal of the release liner 36. Lobes with various release liner 36 and tab 38 configurations can been seen in FIGS. 8a-8c. The lobe 28 with the perforated section is shown in FIG. 8a, with an optional two tab 38 options depicted in FIG. 8b. These embodiment will allow the lobes 28 or the full adhesive pad 12 to be removed from the guidance device 10 or rigid ring 34, and replaced with fresh adhesive for additional use and/or placement of the guidance device 10. FIG. 8c depict the lobe 28 with the release liner 36 removed to expose the adhesive 16.

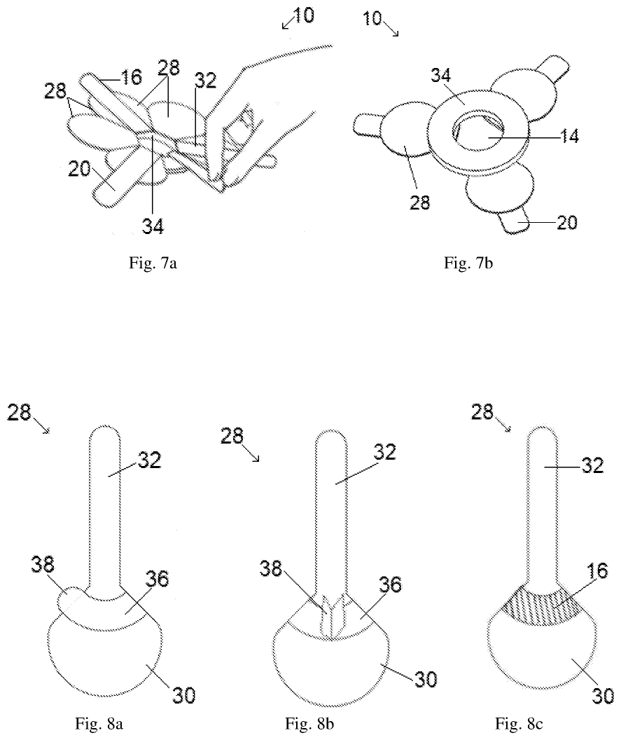

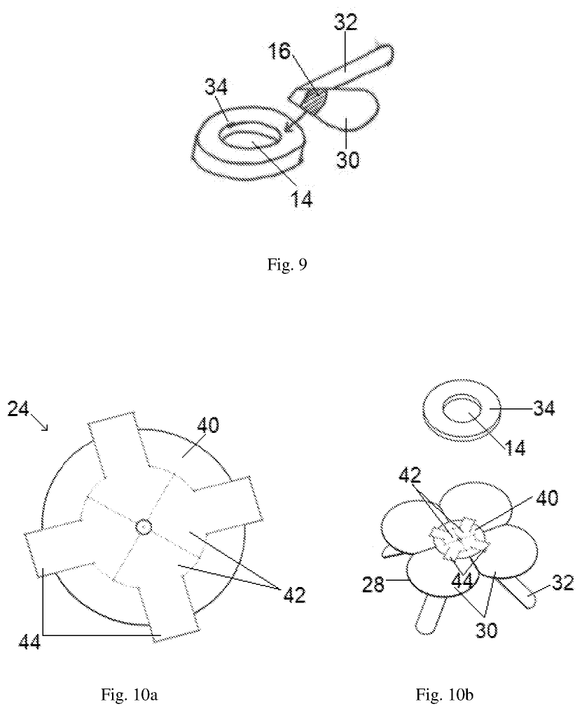

An example of the subject guidance device 10, utilizing six lobes 28 is provided in FIGS. 7a and 7b. FIG. 7a depicts the guidance device 10 with all six lobes 28 attached, wherein FIG. 7b depict the guidance device 10 after three lobes 28 have been used and discarded.

In another embodiment, the transparent target 24 may be made from a sterile, pierceable material. The material may or may not have markings to indicate the point of the target. There may or may not be an opening in the targeted area of the material. This target 24 will be permanently connected between the adhesive pad 12 or lobes 28, and the guidance device 10 and/or rigid ring 34. The connection may be achieved through adhesive, heat bonding, or mechanical hardware.

In another embodiment, the transparent target 24 will be perforated into sections in-line with the perforations separating with the at least two lobes 28 as shown in FIGS. 10a and 10b. The outer circle 40 of the transparent target 24 will be permanently adhered to the device 10, rigid ring 34, or the back of the adhesive pad 12, depending on preferred embodiment. The perforated sections 42 of the transparent target 24 will also have release tabs 44 for removing the respective perforated section 42. In one embodiment, when the tab 20 of the adhesive pad 12, or pull tab 26 for the lobe 28, is pulled to expose the adhesive, the adjoined transparent target 24 release tab 44 may be removed with it.

Thus, in use, there is provided a method for guiding a medical instrument using the medical guidance device 10 as described herein. In a surgical workflow, a user will often mark a target location on a patient where the medical instrument should go, where this target location is based on, for example, prior imaging information about the position of interest within the patient (e.g., a tumor to be biopsied or ablated). The user holds the medical guidance device 10 at a target location on a patient, where this may be simplified by the presence of a transparent target 24 indicating the exact position on the medical guidance device 10 that should correspond with the target location. While the medical guidance device 10 is held at the target location, the user mounts the medical guidance device 10 using the adhesive 16. To do this, the user simply pulls the tabs 20 from the underside of the adhesive pads 12 while maintaining the position of the medial guidance device 10 on the patient. Dependent on whether there will be a need to reposition the device as well as how firmly the user wants to mount the medical guidance device 10, all or a subset of the tabs 20 will be pulled to adhere the medical guidance device 10 onto the patient. After the medical guidance device 10 is mounted, the user can guide a medical instrument to a position from the target location.

The present application hereby incorporates by reference, in their entirety, U.S. Pat. No. 9,222,996 titled “Needle Placement Manipulator with Two Rotary Guides”, and United States Patent Publication No. 2018/0228568 titled “Medical Guidance Apparatus”. In particular, the medical guidance device as described herein may include the described needle placement manipulator or the medical guidance apparatus as the guidance portion and base and thus may be fixed on a patient using the tabs and release liner as described herein.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A mountable medical guiding device comprising:
an adhesive pad having at least two sections of adhesive; and
at least two tabs each releasable attached to the adhesive pad and each extending distally from the base,
wherein each of the at least two tabs may be removed for exposing each of the at least two sections of adhesive for attaching the medical guiding device, and
wherein each of the at least two tabs are removable while the medical guiding device is being held in a mounting position on a subject.

2. The medical guidance device according to claim 1, further comprising a transparent target for aligning the device with a target on the subject.

3. The medical guidance device according to claim 1, wherein the adhesive for each of at least two tabs is removably attachable to the adhesive pad.

4. The medical guidance device according to claim 1, wherein each of the at least two tabs are divided into at least two segments.

5. The medical guidance device according to claim 1, wherein each of the two tabs may be removed and/or replaced from the medical guiding device.

6. The medical guidance device according to claim 1, wherein each of the two tabs may have adhesive on both surfaces of the tab.

7. The medical guidance device according to claim 1, further comprising a guide removably attached to the medical guiding device.

8. The medical guidance device according to claim 1, further comprising a guide permanently attached to the medical guiding device.

9. The medical guidance device according to claim 1, wherein the base comprises a rigid ring that provides structural support to the medical guiding device.

10. The medical guidance device according to claim 2, wherein the transparent target includes a visual or mechanical element.

11. The medical guidance device according to claim 2, wherein the transparent target is removable.

12. The medical guidance device according to claim 2, wherein the transparent target is pierceable.

13. A medical guiding device comprising:
an adhesive pad;
a guidance portion configured to guide a medical instrument; and
at least two tabs each releasable attached to the adhesive pad and each extending distally from the adhesive pad,
wherein each of the at least two tabs may be removed for exposing an adhesive for attaching the device,
wherein each of the at least two tabs is segmented, allowing for partial or complete exposure of the adhesive while the medical guiding device is held in a mounting position on the patient.

14. A method for guiding a medical instrument, the method comprising:
providing a medical guiding device comprising:
an adhesive pad;
a guidance portion; and
at least two tabs each releasable attached to the adhesive pad and each extending distally from the adhesive pad, wherein each of the at least two tabs may be removed for exposing an adhesive for attaching the device, and wherein at least one of the at least two tabs is segmented, allowing for partial or complete exposure of the adhesive,
holding the medical guidance device at a target location on a patient;
mounting the medical guidance device at the target location by removing at least one segment of the at least two tabs without substantially moving the medical guiding device; and
guiding a medical instrument to a position from the target location.

15. The method of claim 14, further comprising
aligning a transparent target located on the medical guidance device with the target location on a patient prior to holding the medical guidance device at the target location, and
removing the transparent target from the medical guidance device before guiding a medical instrument to a position on the desired target.
